(19) United States
(12) Patent Application Publication
(10) Pub. No.: US 2026/0153490 A1
(43) Pub. Date: Jun. 4, 2026

(54) SYSTEMS AND METHODS FOR GAS EMERGENCY MANAGEMENT IN URBAN PUBLIC SPACES BASED ON IOT LARGE MODELS

(71) Applicant: CHENGDU QINCHUAN IOT TECHNOLOGY CO., LTD., Chengdu (CN)

(72) Inventors: Hanshu SHAO, Chengdu (CN); Yong LI, Chengdu (CN); Guobin LUO, Chengdu (CN)

(73) Assignee: CHENGDU QINCHUAN IOT TECHNOLOGY CO., LTD., Chengdu (CN)

(21) Appl. No.: 19/460,335

(22) Filed: Jan. 27, 2026

(30) Foreign Application Priority Data
Oct. 22, 2025 (CN) 202511508370.0

(51) Int. Cl. G01N 33/00; G06V 10/25; G06V 20/52; H05B 47/105

(52) U.S. Cl. CPC G01N 33/0065; G06V 10/25; G06V 20/52; H05B 47/105

(57) ABSTRACT

A system and a method for gas emergency management in an urban public based on a IoT large model are provided. The method includes: obtaining gas monitoring data of a target region; in response to an evacuation condition being satisfied; generating an evacuation parameter based on the gas monitoring data and region data of the target region; and based on the evacuation parameter, sending an evacuation control signal to an evacuation device deployed in the target region to control the evacuation device to guide an evacuation of personnel in the target region. Guiding the evacuation of personnel includes: controlling a display device to display the evacuation path; controlling a display color and an indication direction of an electronic indicator sign; controlling a lighting brightness and a lighting mode of a lighting device; and controlling an on-off state of a gate.

### CROSS-REFERENCE TO RELATED APPLICATIONS

This application claims priority to Chinese Patent Application No. 202511508370.0, filed on Oct. 22, 2025, the entire contents of each of which are hereby incorporated by reference.

### TECHNICAL FIELD

The present disclosure generally relates to a field of gas emergency management in public places, and in particular to systems and methods for gas emergency management in urban public spaces based on Internet of Things (IoT) large models.

### BACKGROUND

In the process of urban development, safety management of public places faces a plurality of bottlenecks. In personnel-intensive public places such as scenic spots, squares, and transportation hubs, gas leakage may cause evacuation difficulties due to complex spaces and dense crowds, thereby leading to regional stampede risks and secondary disaster hazards.

Therefore, it is desirable to provide a system and a method for gas emergency management in an urban public space based on an IoT large model. The system and method can formulate targeted evacuation plans for different environments and different gas leakage situations, send evacuation control signals to evacuation devices, and control the evacuation devices to guide personnel to evacuate quickly and effectively.

### SUMMARY

One or more embodiments of the present disclosure provide a system for gas emergency management in an urban public space based on an IoT large model. The system includes an emergency supervision management platform and an emergency supervision object platform. The emergency supervision management platform is configured to execute a method for gas emergency management in an urban public space based on an IoT large model.

One or more embodiments of the present disclosure provide a method for gas emergency management in an urban public space based on an IoT large model. The method is executed by an emergency supervision management platform of a system for gas emergency management in an urban public space based on the IoT large model. The method includes: obtaining gas monitoring data of a target region through a monitoring device deployed in a monitoring region based on an emergency supervision object platform; in response to an evacuation condition being satisfied, generating an evacuation parameter based on the gas monitoring data and region data of the target region, the evacuation parameter including an evacuation path, an indicator sign control parameter, a lighting control parameter, and a gate control parameter; and sending an evacuation control signal to an evacuation device deployed in the target region based on the evacuation parameter, to control the evacuation device to guide an evacuation of personnel in the target region, including: controlling a display device to display the evacuation path; controlling a display color and an indication direction of an electronic indicator sign based on the indicator sign control parameter; controlling a lighting brightness and a lighting mode of a lighting device based on the lighting control parameter; and controlling an on-off state of a gate based on the gate control parameter.

One or more embodiments of the present disclosure provide a non-transitory computer-readable storage medium, storing one or more computer instructions, wherein when a computer reads the one or more computer instructions in the storage medium, the computer executes a method for gas emergency management in an urban public space based on an IoT large model.

### BRIEF DESCRIPTION OF THE DRAWINGS

The present disclosure is further described in an illustrative manner by way of exemplary embodiments, which are described in detail with reference to the accompanying drawings. These embodiments are not restrictive. In these embodiments, the same reference numerals denote the same structures, wherein:

FIG. 1 is a schematic diagram of an exemplary structure of a system for gas emergency management in an urban public space based on an IoT large model according to some embodiments of the present disclosure;

FIG. 2 is a flowchart of an exemplary process of a method for gas emergency management in an urban public space based on an IoT large model according to some embodiments of the present disclosure;

FIG. 3 is a flowchart of an exemplary process for determining an evacuation path according to some embodiments of the present disclosure;

FIG. 4 is a flowchart of an exemplary process for updating a safe region and one or more candidate evacuation paths according to some embodiments of the present disclosure; and

FIG. 5 is a schematic diagram of the determination of a modality parameter according to some embodiments of the present disclosure.

### DETAILED DESCRIPTION

Drawings required for describing the embodiments are briefly introduced below. The drawings do not represent all embodiments.

In the embodiments of the present disclosure, when operations performed are described step by step, unless otherwise specified, the order of the steps is adjustable, a step may be omitted, and other steps may be included in the processes.

As indicated in the present disclosure and in the claims, the singular forms “a,” “an,” and “the” may be intended to include the plural forms as well, unless the context clearly indicates otherwise. In general, the terms “comprise,” “comprises,” and/or “comprising,” “include,” “includes,” and/or “including,” when used in this disclosure, specify the presence of stated features, integers, steps, operations, elements, and/or components, but do not preclude the presence or addition of one or more other features, integers, steps, operations, elements, components, and/or groups thereof.

The embodiments in the present disclosure are merely for illustration and description, and do not limit the scope of application of the present disclosure. For those skilled in the art, various modifications and changes that may be made under the guidance of the present disclosure still fall within the scope of the present disclosure. In addition, certain features, structures, or characteristics in one or more embodiments of the present disclosure may be appropriately combined.

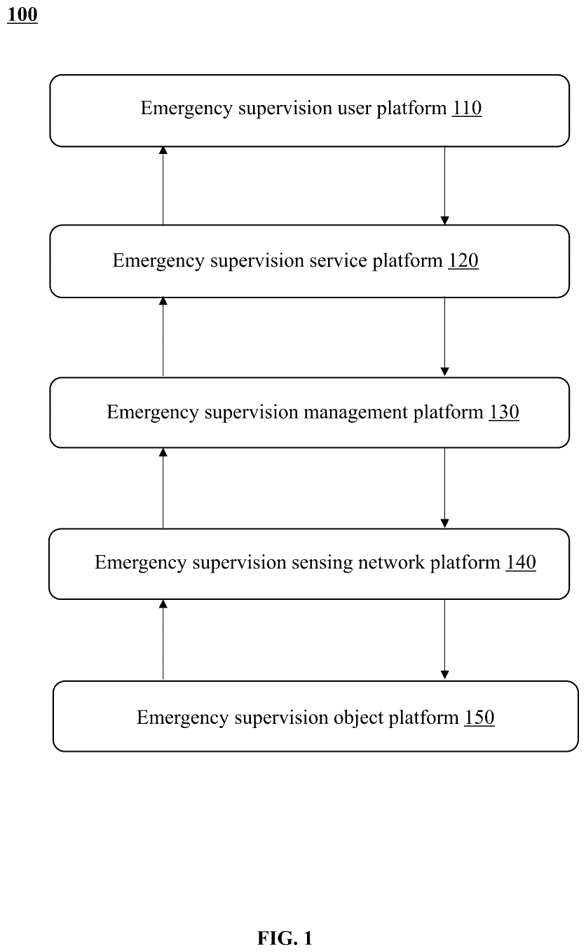

FIG. 1 is a schematic diagram of an exemplary structure of a system for gas emergency management in an urban public space based on an IoT large model according to some embodiments of the present disclosure.

In some embodiments, as shown in FIG. 1, a system 100 for gas emergency management in an urban public space based on an IoT large model (also referred to as the system 100, the system, or the gas emergency management system) includes an emergency supervision user platform 110, an emergency supervision service platform 120, an emergency supervision management platform 130, an emergency supervision sensing network platform 140, and an emergency supervision object platform 150.

The emergency supervision user platform 110 refers to an interactive platform that provides emergency service operations for users. The emergency supervision user platform 110 includes user terminals, e.g., a computer or other devices with input and/or output functions.

The users include a superior supervision department user and a citizen user. The superior supervision department user refers to a superior management department of the emergency supervision management platform 130. The superior supervision department user may deploy an emergency supervision task to the emergency supervision management platform 130 through the emergency supervision user platform 110 and simultaneously receive emergency supervision dynamic information fed back by the emergency supervision management platform 130. The citizen user refers to a person in a public space who has an evacuation need, for example, a tourist in a shopping mall, a hotel resident, a staff member of a catering enterprise, or the like.

The emergency supervision service platform 120 refers to an interactive service platform that receives and transmits data, and includes a server, a gateway, a router, or the like.

In some embodiments, the emergency supervision service platform 120 interacts upward with the emergency supervision user platform 110 and interacts downward with the emergency supervision management platform 130.

The emergency supervision management platform 130 refers to a comprehensive platform that processes and manages emergency supervision data.

In some embodiments, the emergency supervision management platform includes a processor and/or a server, a data center, or the like. The data center is configured with a storage device.

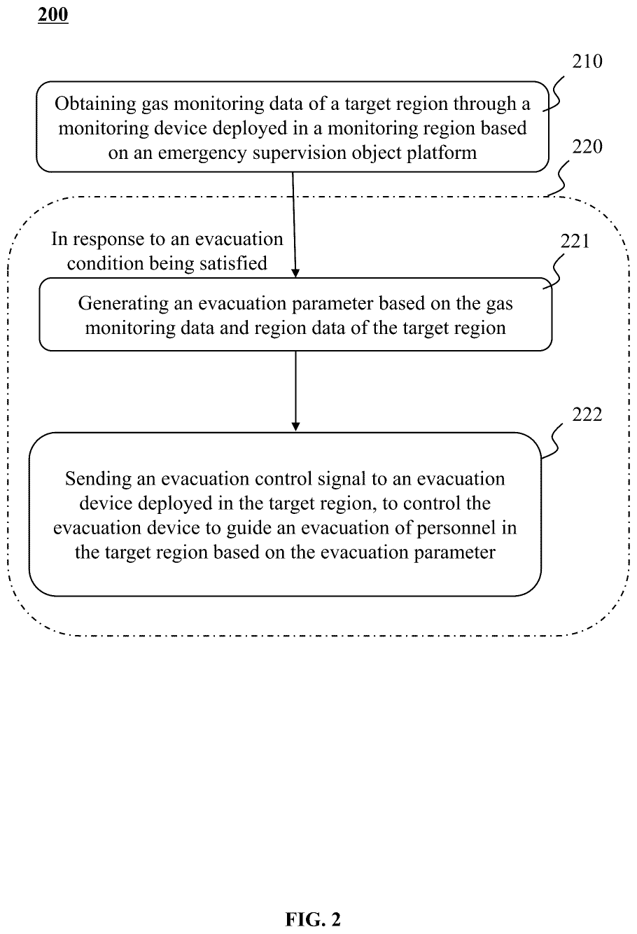

In some embodiments, the emergency supervision management platform 130 is configured to execute a method for gas emergency management in an urban public space based on an IoT large model. More descriptions of the method may be found in FIG. 2 and related descriptions thereof.

The emergency supervision sensing network platform 140 refers to a management platform that transmits emergency supervision-related sensing data or information and includes a communication transmission network and a routing device.

In some embodiments, the emergency supervision sensing network platform 140 interacts upward with the emergency supervision management platform 130 and interacts downward with the emergency supervision object platform 150.

The emergency supervision object platform 150 refers to a platform for collecting emergency supervision data and executing implementation instructions.

In some embodiments, the emergency supervision object platform is configured with a plurality of monitoring devices, storage units, and evacuation devices. The monitoring devices include an image acquisition device, an environment monitoring device, a gas monitoring device, or the like.

The image acquisition device refers to a device for obtaining image data, e.g., an electronic camera or the like. The image data includes image sensing data, external image data, or the like.

The environment monitoring device refers to a device for monitoring environment data, e.g., a temperature sensor, a humidity sensor, a wind direction detector, or the like.

The gas monitoring device refers to a device for monitoring gas data, e.g., a gas concentration monitoring device or the like.

The evacuation device refers to a device that provides evacuation support in an emergency. In some embodiments, the evacuation device includes a gate, an electronic indicator sign, a lighting device, a display device, a ventilation device, a projection device, a broadcast device, or the like.

The electronic indicator sign is used to guide a direction or provide a prompt. The display device is used to present visual information, e.g., an evacuation path. The ventilation device is used to evacuate harmful gas to reduce the gas concentration in air. For example, the ventilation device includes a fan, an exhaust fan, an air purifier, or the like. The projection device is configured to project an image to guide a direction. For example, the projection device projects an evacuation path, or the like. The broadcast device is used to transmit audio information. For example, the broadcast device broadcasts the evacuation path, or the like.

In some embodiments, the emergency supervision object platform is further configured with a sensing device, e.g., a light sensor, a sound level detector, or the like. The sensing device is configured to obtain environmental perception data. For example, the light sensor detects environmental light intensity, and the sound level detector monitors environmental sound intensity.

In some embodiments, the system 100 can establish an information operation closed loop among various functional platforms, operating in a coordinated and regular manner. By efficiently and accurately determining actual evacuation control parameters, the system aims to improve emergency evacuation efficiency and prevent the occurrence of stampede accidents.

FIG. 2 is a flowchart of an exemplary process of a method for gas emergency management in an urban public based on an IoT large model according to some embodiments of the present disclosure. In some embodiments, the method is performed by an emergency supervision management platform (e.g., the emergency supervision management platform 130). As shown in FIG. 2, a process 200 includes the following steps.

Step 210, gas monitoring data of a target region is obtained through a monitoring device deployed in a monitoring region based on an emergency supervision object platform (e.g., the emergency supervision object platform 150).

The monitoring region refers to a region that requires safety monitoring and control. In some embodiments, the monitoring region includes the target region and an external environment region.

The target region refers to a region with a demand for gas safety evacuation. For example, the target region includes interior areas of the public space that use gas, such as shopping malls, hotels, catering enterprises, or the like.

The external environment region refers to a surrounding environment outside the target region. For example, the external environment region includes roads, buildings, or the like, around the target region.

The gas monitoring data refers to parameters related to gases in the target region. For example, the gas monitoring data includes a gas concentration, an oxygen concentration, or the like in the target region. The gas concentration is represented by a concentration of a harmful gas (e.g., methane).

In some embodiments, the gas monitoring data is obtained through a gas monitoring device.

In some embodiments, in response to an evacuation condition being satisfied, the emergency supervision management platform performs step 220. The step 220 includes step 221 and step 222.

The evacuation condition refers to a critical condition that triggers personnel evacuation when a gas leakage hazard occurs.

In some embodiments, the evacuation condition includes the gas concentration in the gas monitoring data being higher than a first preset concentration threshold. The first preset concentration threshold is predetermined manually based on experience or set by default by a processor.

Step 221, an evacuation parameter is generated based on the gas monitoring data and region data of the target region.

The region data refers to data describing a situation of the target region. For example, the region data includes terrain data in the target region, positions of passages, passage dimensions, or the like. The region data is obtained based on a plan view and/or a three-dimensional view of the target region.

The evacuation parameter refers to a parameter for guiding personnel evacuation, including an evacuation path, an indicator sign control parameter, a lighting control parameter, and a gate control parameter.

The evacuation path refers to a passage route for personnel evacuation.

The indicator sign control parameter refers to an operation parameter of an electronic indicator sign for guiding personnel evacuation. For example, the indicator sign control parameter includes an indication direction and a display color of the electronic indicator sign.

The lighting control parameter refers to an operating parameter of a lighting device for guiding personnel evacuation. For example, the lighting control parameter includes a lighting brightness of the lighting device, and a lighting mode of the lighting device. The lighting mode includes a constant intensity mode, a flashing mode, or the like.

The gate control parameter refers to an operating parameter of a gate for guiding personnel evacuation. For example, the gate control parameter includes an on-off state of the gate.

In some embodiments, the emergency supervision management platform generates a plurality of connected paths based on the region data of the target region through a path planning algorithm. The emergency supervision management platform selects one or more connected paths from the plurality of connected paths as evacuation path(s) according to a selection condition. For example, the selection condition may be a shortest path length or a highest path score.

Connected paths refer paths connecting an interior of the target region to an external environment region. Path scores may be represented by a first weighted sum of normalized path lengths and exit capacities. The exit capacity of a connected path refers to a capacity at an exit corresponding to the connected path. The exit capacity of each region is preset manually. The path score of each connected path is determined based on a region where the exit of the connected path is located. The path planning algorithm may be a Dijkstra algorithm. The normalization manner may be Min-Max normalization.

In some embodiments, the emergency supervision management platform determines the indicator sign control parameter, the lighting control parameter, and the gate control parameter based on the evacuation path.

For example, the emergency supervision management platform uses a direction of the evacuation path as an indication direction of the electronic indicator sign on the evacuation path, and uses a preset emergency display color (e.g., red) as a display color. The emergency supervision management platform uses a first preset brightness as the lighting brightness of a lighting device on the evacuation path and sets the lighting mode of the lighting device to the flashing mode. The emergency supervision management platform uses a second preset brightness as the lighting brightness of a lighting device not on the evacuation path and sets the lighting mode of the lighting device to the constant intensity mode. The emergency supervision management platform opens the gate on the evacuation path and closes the gate not on the evacuation path. The first preset brightness is higher than the second preset brightness. The first preset brightness, the second preset brightness, and the preset emergency display color may be preset manually.

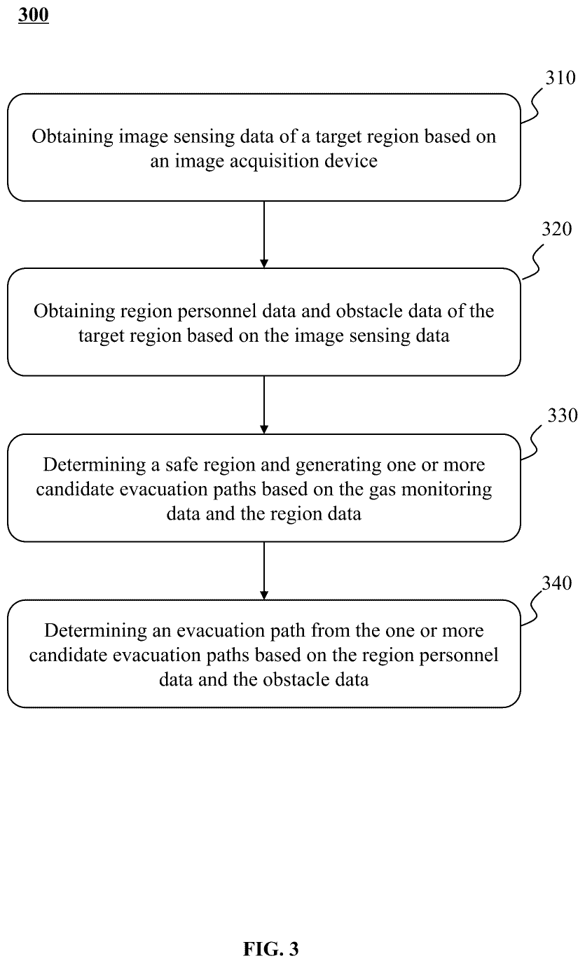

In some embodiments, the emergency supervision management platform obtains image sensing data of the target region, thereby obtaining region personnel data and obstacle data of the target region. The emergency supervision management platform determines a safe region and generates one or more candidate evacuation paths based on the gas monitoring data and the region data. The emergency supervision management platform determines the evacuation path from the one or more candidate evacuation paths based on the region personnel data and the obstacle data. More descriptions regarding the determination of the evacuation path may be found in FIG. 3 and related descriptions thereof.

Step 222, an evacuation control signal is sent to an evacuation device deployed in the target region to control the evacuation device to guide an evacuation of personnel in the target region based on the evacuation parameter.

The evacuation control signal refers to a signal used to control the evacuation device to operate according to the evacuation parameter during an evacuation process.

In some embodiments, the evacuation control signal is used to control the evacuation device to guide the evacuation of personnel in the target region. Controlling the evacuation device includes controlling a display device to display the evacuation path; based on the indicator sign control parameter, controlling the display color and the indication direction of the electronic indicator sign; based on the lighting control parameter, controlling the lighting brightness and the lighting mode of the lighting device; and based on the gate control parameter, controlling the on-off state of the gate.

In some embodiments, in response to reaching an evacuation condition, the evacuation parameter is generated through coordination of the gas monitoring data and the region data. The evacuation parameter is used to control the evacuation device to guide the evacuation of personnel in the target region. This approach can effectively improve crowd evacuation efficiency, reduce the risk of crowding and stampedes, enhance emergency response capabilities, and ensure public safety. Precise adjustment of the indicator sign control parameter can enhance dynamic adaptability of directional signs and meet guidance requirements of different scenarios. Adjustment of the lighting control parameter can ensure visibility in low-visibility environments and further enhance guidance of the path. Intelligent control of the on-off state of the gate achieves precise coordination of physical isolation and path management, effectively preventing personnel from mistakenly entering a hazardous region. The entire set of control signal linkage mechanisms, through parametric instruction transmission, ensures the reliability and response speed of multi-device collaborative operations, significantly improving the emergency response effectiveness for public safety incidents in complex environments.

In some embodiments, the evacuation parameter further includes a ventilation control parameter.

In some embodiments, the emergency supervision management platform controls an air supply intensity and an air supply direction of a ventilation device based on the ventilation control parameter.

The ventilation control parameter refers to a parameter used to control operation of the ventilation device. The ventilation control parameter includes the air supply direction and the air supply intensity.

In some embodiments, the emergency supervision management platform determines the ventilation control parameter based on the gas monitoring data and the region data. For example, the emergency supervision management platform generates the evacuation path based on the gas monitoring data and the region data, determines a direction of decreasing gas concentration as a gas diffusion direction based on the gas monitoring data and determines a diffusion speed of the gas as a gas leakage speed. The emergency supervision management platform further activates the ventilation device located on the evacuation path and determines a direction opposite to the gas diffusion direction as the air supply direction of the ventilation device. Subsequently, the emergency supervision management platform performs a second weighted sum on a normalized gas leakage speed and a normalized evacuation path length. The air supply intensity of the ventilation device is positively correlated with a result of the second weighted sum. Related descriptions about how to generate the evacuation path based on the gas monitoring data and the region data may be found in step 221 and related descriptions thereof.

In some embodiments, by automatically starting the ventilation device in a corresponding region to supply air to the evacuation path, the intrusion of toxic, harmful gases, or smoke is effectively prevented, ensuring the safety and unobstructed flow of the evacuation route.

FIG. 3 is a flowchart of an exemplary process for determining an evacuation path according to some embodiments of the present disclosure. In some embodiments, the determination of the evacuation path is performed by the emergency supervision management platform. As shown in FIG. 3, a process 300 includes the following steps.

Step 310, based on an image acquisition device, image sensing data of a target region is obtained.

The image sensing data refers to image data monitored and acquired by the image acquisition device.

Step 320, based on the image sensing data, region personnel data and obstacle data of the target region is obtained.

The region personnel data refers to data describing personnel conditions in a target region. For example, the region personnel data may include a personnel count, a personnel density, and a personnel movement direction.

In some embodiments, the emergency supervision management platform, based on the image sensing data, identifies and obtains the personnel count, the personnel movement direction, a region area size, or the like through an image recognition algorithm, and determines a ratio of the personnel count to the region area size as the personnel density. The image recognition algorithm may be a Haar feature cascade algorithm.

The obstacle data refers to data describing obstacle conditions in the target region. For example, the obstacle data may include an obstacle size, an obstacle position, an obstacle area proportion, an obstacle count, or the like.

In some embodiments, the emergency supervision management platform, based on the image sensing data, identifies and obtains the obstacle data through an image recognition algorithm.

Step 330, based on gas monitoring data and region data, a safe region is determined, and one or more candidate evacuation paths are generated.

The safe region refers to a region that can effectively avoid hazards from gas. In some embodiments, the safe region refers to a region where a gas concentration is lower than a second preset concentration threshold.

In some embodiments, the emergency supervision management platform, based on terrain data in the region data, divides the target region into a plurality of functional sub-regions. A functional sub-region refers to a sub-region obtained by dividing the target region according to regional functions, for example, a corridor, a stairwell, an elevator, a hall, a room, or the like. Then, the emergency supervision management platform inputs the gas monitoring data and the region data into a Gaussian plume model to output an estimated gas concentration for each functional sub-region. Subsequently, the emergency supervision management platform determines a functional sub-region with an estimated gas concentration lower than the second preset concentration threshold as the safe region, and determines a functional sub-region with an estimated gas concentration higher than a third preset concentration threshold as a high-risk region.

The second preset concentration threshold and the third preset concentration threshold may be set manually based on experience. The second preset concentration threshold is less than the first preset concentration threshold, and the first preset concentration threshold is less than the third preset concentration threshold.

A candidate evacuation paths refer to a potential path for determining the final evacuation path.

In some embodiments, the emergency supervision management platform removes connected paths passing through the high-risk region and uses remaining connected path(s) as the candidate evacuation path(s). More descriptions regarding the connected path and the evacuation path may be found in the related description above.

Step 340, based on the region personnel data and the obstacle data, the evacuation path is determined from the one or more candidate evacuation paths.

In some embodiments, the emergency supervision management platform, based on the region personnel data and the obstacle data, determines an obstacle impact degree and a congestion degree of the candidate evacuation path; based on the region data, determines a path length and a path complexity of the candidate evacuation path; based on the path complexity, the path length, the obstacle impact degree, and the congestion degree of the candidate evacuation path, determines an evacuation difficulty level by querying an evacuation difficulty table; and selects N candidate evacuation paths with the smallest evacuation difficulty level as evacuation paths. The evacuation difficulty table is constructed based on historical data or experimental data. A higher path complexity, a longer path length, a greater obstacle impact degree, and a higher congestion degree of a candidate evacuation path all lead to a greater evacuation difficulty level of the candidate evacuation path.

For example, the emergency supervision management platform determines the obstacle area proportion and the obstacle count on the candidate evacuation path; performs a third weighted summation on a normalized obstacle area proportion and a normalized obstacle count to obtain the obstacle impact degree of the candidate evacuation path. The congestion degree of the candidate evacuation path is positively correlated with the personnel density. The emergency supervision management platform further determines a ratio of a straight-line distance between a start point and an end point of the candidate evacuation path to the path length, and determines a product of the ratio and a curvature weight as the path complexity of the candidate evacuation path.

The curvature weight is positively correlated with the count of right-angle turns in the candidate evacuation path, the value of N is positively correlated with the personnel count in the target region, and weights for the third weighted summation are preset manually based on experience. The count of right-angle turns is obtained based on the candidate evacuation path through a corner detection algorithm, e.g., a Harris corner detection algorithm.

In some embodiments, the emergency supervision management platform, based on the image acquisition device, obtains external image data; based on the region personnel data, the obstacle data, and the region data, determines an evacuation efficiency of each of the one or more candidate evacuation paths; and, based on the external image data and the evacuation efficiency, determines the evacuation path from the one or more candidate evacuation paths.

The external image data refers to image data of an external environment region acquired by the image acquisition device.

The evacuation efficiency of a candidate evacuation paths refers to the count of persons (the personnel count) evacuated per unit time through the candidate evacuation path, which is used to measure the evacuation capability of the candidate evacuation path.

In some embodiments, the emergency supervision management platform, based on the region personnel data, the obstacle data, and the region data of the functional sub-regions that the candidate evacuation path pass through, constructs a first target feature vector; performs a search in a first vector database to retrieve the first target feature vector to determine a first reference feature vector having the highest vector similarity with the first target feature vector; and determines a first reference evacuation efficiency corresponding to the first reference feature vector as the evacuation efficiency of the candidate evacuation path. The first vector database includes a plurality of first reference feature vectors and their corresponding first reference evacuation efficiencies, and is constructed based on historical data. For example, the first reference feature vector is constructed based on historical region personnel data, historical obstacle data, and historical region data of each functional sub-region passed by a historical evacuation path, and the first reference evacuation efficiency is represented by the actual count of persons evacuated per unit time through the historical evacuation path. The vector similarity may be measured by metrics such as cosine similarity, Euclidean distance, or the like.

In some embodiments, the emergency supervision management platform, based on the region personnel data, the obstacle data, the region data, and access control data, determines the evacuation efficiency of each of the one or more candidate evacuation paths.

The access control data refers to data related to an access control device. The access control device includes a gate. The access control data includes a location of the gate, an opening speed of the gate, a maximum throughput of the gate, an opening size of the gate, or the like.

In some embodiments, the access control data is obtained by manual input.

In some embodiments, the emergency supervision management platform constructs a second target feature vector based on the region personnel data, the obstacle data, the region data, and the access control data of the functional sub-regions that the candidate evacuation path pass through. Then the emergency supervision management platform retrieves the second target feature vector in a second vector database to determine a second reference feature vector with the highest vector similarity to the second target feature vector. Subsequently, the emergency supervision management platform determines a second reference evacuation efficiency corresponding to the second reference feature vector as the evacuation efficiency of the candidate evacuation path. The second vector database includes a plurality of second reference feature vectors and second reference evacuation efficiencies corresponding to the second reference feature vectors. The construction manner of the second vector database is similar to that described for the first vector database and will not be reiterated here.

In some embodiments, the evacuation efficiency of each path can be calculated more accurately by considering the access control data. This facilitates rapid matching of an optimal evacuation plan by the system, shortens decision-making time, and improves decision-making accuracy.

In some embodiments, for each candidate evacuation path, the emergency supervision management platform identifies and obtains a personnel density, a vehicle density, and an environment type (e.g., a lane, a square, or the like) of the external environment region based on external image data by using an image recognition algorithm; determines a correction coefficient by querying a correction coefficient lookup table based on the environment type, the personnel density, and the vehicle density of the external environment region; and determines a product of the evacuation efficiency and the correction coefficient as a corrected evacuation efficiency of the candidate evacuation path. The emergency supervision management platform sorts a plurality of candidate evacuation paths in descending order according to their corrected evacuation efficiencies. The emergency supervision management platform selects the top M candidate evacuation paths as evacuation paths. The value of M and N may be the same or different.

The correction coefficient lookup table is determined manually based on historical data or experimental data. In some embodiments, the emergency supervision management platform determines a ratio of the personnel quantity in the target region to a preset evacuation time as a required evacuation efficiency. Then the emergency supervision management platform sorts the plurality of candidate evacuation paths in descending order according to their corrected evacuation efficiencies, and sequentially accumulates and sums the corrected evacuation efficiencies corresponding to the sorted candidate evacuation paths to obtain a total evacuation efficiency. A minimum count of candidate evacuation paths required for the total evacuation efficiency to be greater than the required evacuation efficiency is taken as M. The required evacuation efficiency refers to a minimum evacuation efficiency necessary for the safe evacuation of personnel within the target region, and the preset evacuation time is set manually based on experience.

In some embodiments, by comprehensively evaluating both the internal evacuation paths within the building and the external environmental conditions at the exits, the risk of congestion caused by insufficient evacuation path capacity can be effectively reduced, thereby enhancing the safety and efficiency of the evacuation process.

In some embodiments, by comprehensively generating candidate evacuation paths based on the gas monitoring data and the region data, and then screening the final evacuation path(s) using actual region personnel data and obstacle data, a balance is effectively achieved between ensuring evacuation efficiency and maintaining the feasibility and rationality of the evacuation path(s). This avoids problems such as congestion due to an insufficient count of evacuation paths or chaos caused by selecting inappropriate evacuation passages.

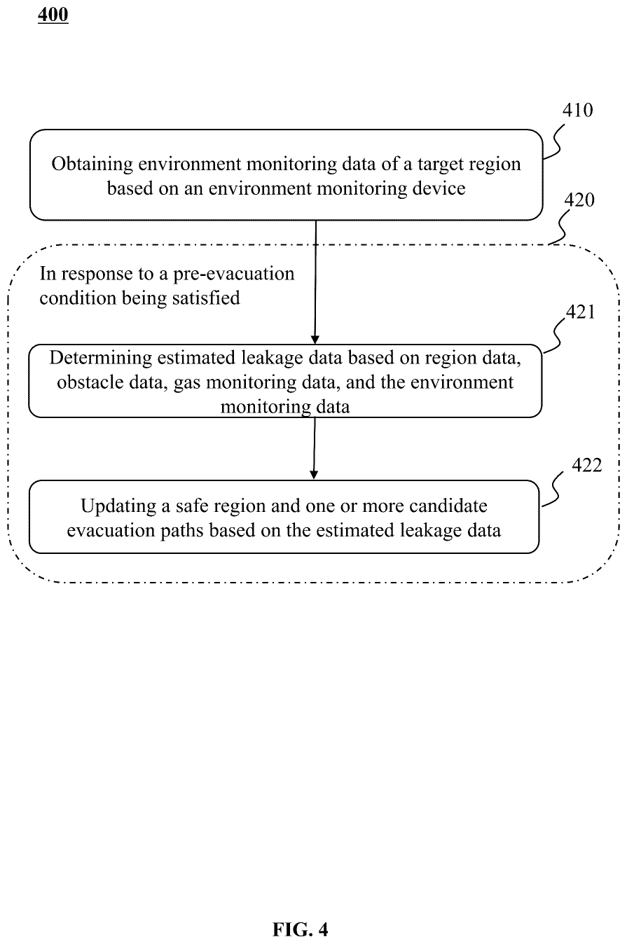

FIG. 4 is a flowchart of an exemplary process for updating a safe region and one or more candidate evacuation paths according to some embodiments of the present disclosure. In some embodiments, the updating of the safe region and the candidate evacuation path is performed by the emergency supervision management platform. As shown in FIG. 4, a process 400 includes the following steps.

Step 410, environment monitoring data of a target region is obtained based on an environment monitoring device.

The environment monitoring data refers to data related to an environmental condition obtained by the environment monitoring device. For example, the environment monitoring data includes an ambient temperature, an ambient humidity level, wind direction data, or the like.

In some embodiments, the emergency supervision management platform executes step 420 in response to a pre-evacuation condition being satisfied. The step 420 includes step 421 and step 422.

The pre-evacuation condition refers to a condition that triggers a pre-warning when a gas leakage hazard occurs. In some embodiments, the pre-evacuation condition refers to a condition where an evacuation condition is not yet met but a warning is required.

In some embodiments, the pre-evacuation condition includes the gas concentration in the gas monitoring data being higher than a fourth preset concentration threshold. The fourth preset concentration threshold is preset manually based on experience. The fourth preset concentration threshold is lower than the first preset concentration threshold. More descriptions regarding the evacuation condition, the gas monitoring data, and the first preset concentration threshold may be found in FIG. 2 and related descriptions thereof.

Step 421, based on the region data, the obstacle data, the gas monitoring data, and the environment monitoring data, estimated leakage data is determined.

More descriptions regarding the region data, the obstacle data, the gas monitoring data may be found in related descriptions above. The estimated leakage data refers to prediction data regarding a gas leakage situation in the target region. In some embodiments, the estimated leakage data is a sequence composed of gas concentrations at a plurality of future time points within a future time period for a plurality of functional sub-regions of the target region. The future time period and the future time points are preset manually based on actual situations.

In some embodiments, the emergency supervision management platform determines the estimated leakage data through a safe region determination model based on the region data, the obstacle data, the gas monitoring data, and the environment monitoring data.

In some embodiments, the safe region determination model is a machine learning model. For example, the safe region determination model is a graph neural network (GNN) model.

In some embodiments, an input of the safe region determination model includes a leakage feature graph. An output of the safe region determination model is estimated leakage data for each node of the leakage feature graph.

The leakage feature graph refers to a graph describing the gas leakage situation in the target region. The leakage feature graph is composed of at least one node and at least one edge.

In some embodiments, one node corresponds to one functional sub-region of the target region. A node attribute of a node includes region data, obstacle data, gas monitoring data, environment monitoring data, a gas leakage speed, and wind direction data of the functional sub-region corresponding to the node. More descriptions regarding the functional sub-region, the region data, the gas monitoring data, the gas leakage speed, and the wind direction data may be found in FIG. 2 and related descriptions thereof.

In some embodiments, the edges of the leakage feature graph may be physical connection edges. An edge exists between two nodes when two functional sub-regions corresponding to the two nodes have a spatial physical connection. An attribute of an edge includes connection data of the two functional sub-regions connected by the edge.

The connection data includes a connection mode, a connection type, and a connection size of the spatial physical connection. The connection mode of the spatial physical connection includes a direct connection and an indirect connection. The direct connection refers to a connection mode where two functional sub-regions exchange gas through a tangible passage, e.g., a door, a window, or the like. The indirect connection refers to a connection mode where two functional sub-regions exchange gas through manners other than tangible passages, e.g., a ventilation system, or the like. The connection type of the spatial physical connection refers to a type of a device for gas exchange between two functional sub-regions, e.g., a bidirectional door, a sliding window, or the like. The connection size of the spatial physical connection refers to a size of the device for gas exchange between two functional sub-regions, e.g., a size of a one-way door, or the like.

In some embodiments, the safe region determination model is obtained by training with a large number of first training samples with first labels. A first training sample includes a sample leakage feature graph constructed based on historical data collected at a first historical time point. The first label may be historical leakage data at a plurality of second historical time points within a second historical period for each node in the sample leakage feature graph. The first historical time point is before the second historical period. The first training samples may be constructed manually based on historical data. The first labels may be annotated manually based on historical data.

In some embodiments, the emergency supervision management platform trains to obtain a safe region determination model based on the first training samples and the first labels. Training manners include, but are not limited to, gradient descent, or the like. Merely by way of example, the emergency supervision management platform inputs a plurality of first training samples into an initial safe region determination model, constructs a loss function based on the first labels and output results of the initial safe region determination model, and iteratively updates parameters of the initial safe region determination model based on the loss function. Model training is completed when a preset condition is satisfied, and a trained safe region determination model is obtained. The preset condition includes convergence of the loss function, an iteration count reaching a preset count threshold, or the like.

Step 422, based on the estimated leakage data, the safe region and the candidate evacuation path are updated.

In some embodiments, the emergency supervision management platform determines a functional sub-region where the gas concentrations at a plurality of future time points in the estimated leakage data are all lower than the second preset concentration threshold and which has no continuous leakage sources, as an updated safe region. For more descriptions of the second preset concentration threshold, reference may be made to the above related descriptions.

In some embodiments, the emergency supervision management platform determines a candidate evacuation path that passes only through updated safe region(s) as an updated candidate evacuation path.

In some embodiments, the emergency supervision management platform updates the safe region and the candidate evacuation path based on the estimated leakage data and ventilation device data of the target region.

The ventilation device data refers to data related to working parameters of the ventilation device, e.g., a maximum air volume, or the like. In some embodiments, the ventilation device data is manually input in advance.

In some embodiments, the emergency supervision management platform determines the actual gas concentration of the functional sub-region according to the following formula:

$$C = {C}^{′} \times ( 1 - \frac{V}{Q} \times w \times t )$$

In the above formula, C represents the actual gas concentration, C′ represents the estimated leakage data, V represents a volume of the target region, Q represents the maximum air volume of the ventilation device, w represents a correction coefficient, and t represents a ventilation time. The ventilation time t is manually input in advance. The correction coefficient w is manually set based on experience.

The emergency supervision management platform determines a functional sub-region where the actual gas concentration is lower than the second preset concentration threshold and which has no continuous leakage sources, as an updated safe region, and determines a candidate evacuation path that passes through updated safe region(s), as the updated candidate evacuation path. In some embodiments, the emergency supervision management platform may determine one or more updated safe regions and one or more updated candidate evacuation paths.

In some embodiments, by considering the dilution of gas concentration in the space by the ventilation device, the subsequent determination of the evacuation path can better align with the evolving real-world situation during the evacuation process.

In some embodiments, selecting an evacuation path by considering gas concentrations throughout the space helps to a certain extent to avoid secondary harm that might occur during the evacuation process, thereby enhancing the safety and effectiveness of the evacuation path.

In some embodiments, the emergency supervision management platform updates an alarm threshold and a monitoring frequency based on the estimated leakage data and one or more updated candidate evacuation paths. Based on an updated alarm threshold, the emergency supervision management platform sends an alarm control signal to an alarm device deployed in the target region to control the alarm device to perform a setting update based on the updated alarm threshold. Furthermore, based on an updated monitoring frequency, the emergency supervision management platform sends a monitoring control signal to an image acquisition device to control the image acquisition device to perform the setting update based on the updated monitoring frequency.

The alarm threshold refers to a gas concentration threshold at which the alarm device triggers an alarm. In some embodiments, the alarm threshold may be identical or similar to the first preset concentration threshold (e.g., with a difference not exceeding 5%).

In some embodiments, the alarm threshold may be preset manually.

The monitoring frequency refers to a frequency at which the image acquisition device performs monitoring.

In some embodiments, when the count of updated candidate evacuation paths is less than a preset count, the emergency supervision management platform calculates a gas leakage rate based on the estimated leakage data using a rate formula, determines a reduction magnitude for the alarm threshold and a reduction magnitude for the monitoring frequency based on the gas leakage rate, and updates the alarm threshold and the monitoring frequency based on the reduction magnitude. The reduction magnitude is positively correlated with the gas leakage rate. The preset count may be preset manually.

In some embodiments, the emergency supervision management platform sends the alarm control signal to the alarm device deployed in the target region based on the updated alarm threshold, to drive the alarm device to perform an alarm threshold update operation. The emergency supervision management platform sends the monitoring control signal to the image acquisition device based on the updated monitoring frequency, to drive the image acquisition device to perform a monitoring frequency update operation.

In some embodiments, the emergency supervision management platform ensures that the system can adapt to changes in environmental conditions through dynamic correction of the alarm threshold and precise regulation of the monitoring frequency of the image acquisition device, thereby achieving reasonable allocation of energy consumption while ensuring evacuation efficiency.

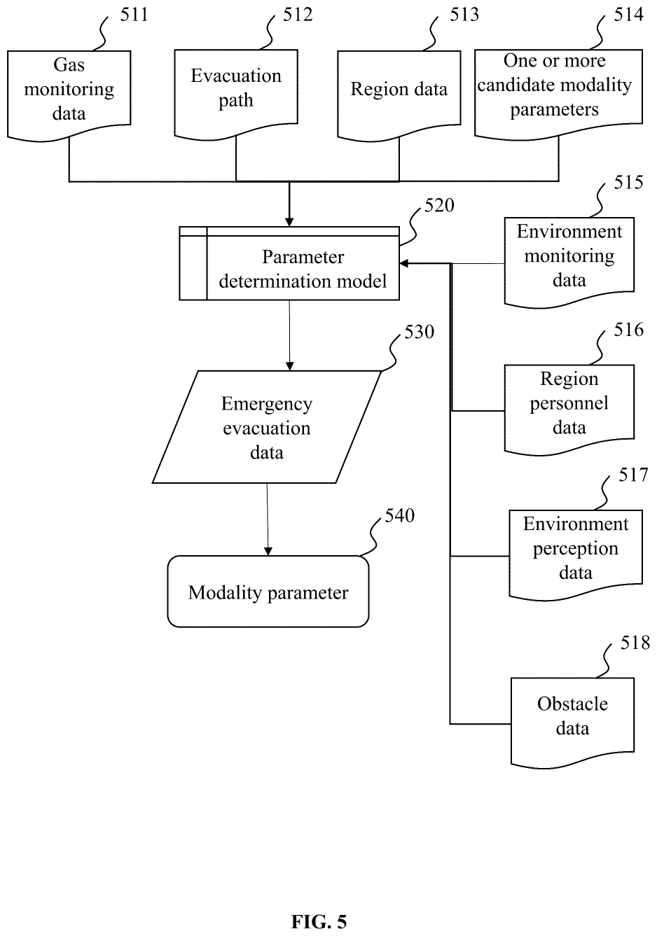

FIG. 5 is a schematic diagram of the determination of a modality parameter according to some embodiments of the present disclosure.

In some embodiments, the evacuation parameter further includes a modality parameter of the evacuation device. As shown in FIG. 5, the emergency supervision management platform obtains one or more candidate modality parameters 514. The emergency supervision management platform determines emergency evacuation data 530 corresponding to each of the one or more candidate modality parameters through a parameter determination model 520 based on an evacuation path 512, region data 513, gas monitoring data 511, and the one or more candidate modality parameters 514. The parameter determination model is a machine learning model. The emergency supervision management platform determines a modality parameter 540 based on the emergency evacuation data 530.

The modality parameter refers to a setting parameter that defines a manifestation form of the evacuation device during evacuation guidance. For example, the modality parameter includes a display brightness of an electronic indicator sign, a flashing frequency of a lighting device, a display brightness of a display device, a projection brightness of a projection device, a volume of a broadcast device, or the like.

A candidate modality parameter refers to a selectable modality parameter during a process of determining the modality parameter. The candidate modality parameter is preset manually or generated randomly by the system.

The emergency evacuation data refers to data describing an estimated evacuation situation of the evacuation path. In some embodiments, the emergency evacuation data includes an evacuation rate and an evacuation completion time of each evacuation path during evacuation.

In some embodiments, the emergency supervision management platform determines, based on the evacuation path, the region data, the gas monitoring data, and the one or more candidate modality parameters, emergency evacuation data corresponding to each of the one or more candidate modality parameters through a parameter determination model.

The parameter determination model is a model for determining the modality parameter.

In some embodiments, the parameter determination model is a machine learning model, e.g., a multimodal fusion model (MFM), or the like.

In some embodiments, as shown in FIG. 5, an input of the parameter determination model includes the evacuation path 512, the region data 513, the gas monitoring data 511, the one or more candidate modality parameters 514, region personnel data 516, environment perception data 517, and obstacle data 518. An output of the parameter determination model is the emergency evacuation data 530 corresponding to each candidate modality parameter 514. More descriptions of the evacuation path, the region data, the region personnel data, the gas monitoring data, and the obstacle data may be found in FIG. 2 and FIG. 3 and the related description.

The environment perception data refers to environment-related data associated with perception. For example, the environment perception data includes an ambient light level, an ambient noise level, etc. In some embodiments, the environment perception data is detected and obtained by a sensing device. For more descriptions of the sensing device, reference may be made to FIG. 1 and related descriptions thereof.

In some embodiments, the parameter determination model is obtained through training using a large number of second training samples with second labels. A second training sample includes a sample evacuation path, sample region data, sample gas monitoring data, a sample modality parameter, sample region personnel data, sample environment perception data, and sample obstacle data. The second training samples may be obtained based on historical data. The second label is actual emergency evacuation data corresponding to the second training sample. The second labels corresponding to the second training samples may be obtained through manual annotation.

A training process of the parameter determination model is similar to the training process of the safe region determination model. Reference may be made to the above description, and details are not repeated herein.

In some embodiments, as shown in FIG. 5, the input of the parameter determination model 520 further includes environment monitoring data 515 of the target region. More descriptions regarding the environment monitoring data may be found in FIG. 4 and related descriptions thereof.

In some embodiments, the second training sample further includes sample environment monitoring data, and the second label may be actual emergency evacuation data corresponding to the second training sample. The manner for obtaining the second training samples and the second labels, and the training process of the parameter determination model, may be referred to in the above descriptions.

In some embodiments, environmental parameters such as temperature and humidity constitute key regulatory factors for a physiological state of a human body. Using the environment monitoring data of the target region as an input variable of the model can quantitatively analyze a dynamic impact of the environment on a path traffic efficiency, and improve prediction accuracy of personnel behavior responses, thereby optimizing effectiveness of an evacuation strategy.

In some embodiments, the emergency supervision management platform selects a candidate modality parameter with a best evacuation effect as the modality parameter. The best evacuation effect means that the candidate modality parameter produces the highest composite score, as determined by a weighted evaluation of the evacuation rate and the evacuation completion time in the emergency evacuation data corresponding to the candidate modality parameter. A faster evacuation rate and a shorter evacuation completion time result in a higher score. The weighting factors may be preset manually.

In some embodiments, by considering multi-dimensional dynamic parameter optimization, evacuation control accuracy and response efficiency in complex emergency scenarios are significantly improved. This approach enhances the system's autonomous adaptability to sudden variable factors, ensuring evacuation safety while optimizing the dynamic allocation efficiency of emergency resources.

The embodiments in the present disclosure are merely for illustration and description, and do not limit the scope of application of the present disclosure. For those skilled in the art, various modifications and changes that can be made under the guidance of the present disclosure are still within the scope of the present disclosure.

In addition, certain features, structures, or characteristics in one or more embodiments of the present disclosure may be appropriately combined.

If a description, a definition, and/or use of a term in the accompanying materials of the present disclosure is inconsistent with or conflicts with the description, definition, and/or use of the term in the present disclosure, the description, definition, and/or use of the term in the present disclosure shall prevail.

What is claimed is:

1. A system for gas emergency management in an urban public space based on an Internet of Things (IoT) large model, comprising: an emergency supervision management platform and an emergency supervision object platform; wherein
the emergency supervision management platform is configured to:
obtain gas monitoring data of a target region through a monitoring device deployed in a monitoring region based on the emergency supervision object platform;
in response to an evacuation condition being satisfied,
generate an evacuation parameter based on the gas monitoring data and region data of the target region, the evacuation parameter including an evacuation path, an indicator sign control parameter, a lighting control parameter, and a gate control parameter; and
send an evacuation control signal to an evacuation device deployed in the target region, to control the evacuation device to guide an evacuation of personnel in the target region based on the evacuation parameter, wherein to control the evacuation device to guide the evacuation of personnel in the target region, the emergency supervision management platform is further configured to:
control a display device to display the evacuation path;
control a display color and an indication direction of an electronic indicator sign based on the indicator sign control parameter;
control a lighting brightness and a lighting mode of a lighting device based on the lighting control parameter; and
control an on-off state of a gate based on the gate control parameter.

2. The system of claim 1, wherein the evacuation parameter further includes a ventilation control parameter, and the emergency supervision management platform is further configured to:
control an air supply intensity and an air supply direction of a ventilation device based on the ventilation control parameter.

3. The system of claim 1, wherein the monitoring device further includes an image acquisition device, and the emergency supervision management platform is further configured to:
obtain image sensing data of the target region based on the image acquisition device;
obtain region personnel data and obstacle data of the target region based on the image sensing data;
determine a safe region and generate one or more candidate evacuation paths based on the gas monitoring data and the region data; and
determine the evacuation path from the one or more candidate evacuation paths based on the region personnel data and the obstacle data.

4. The system of claim 3, wherein the emergency supervision management platform is further configured to:
obtain external image data based on the image acquisition device;
determine an evacuation efficiency of each of the one or more candidate evacuation paths based on the region personnel data, the obstacle data, and the region data; and
determine the evacuation path from the one or more candidate evacuation paths based on the external image data and the evacuation efficiency.

5. The system of claim 4, wherein the emergency supervision management platform is further configured to:
determine the evacuation efficiency of each of the one or more candidate evacuation paths based on the region personnel data, the obstacle data, the region data, and access control data.

6. The system of claim 3, wherein the monitoring device further includes an environment monitoring device, and the emergency supervision management platform is further configured to:
obtain environment monitoring data of the target region based on the environment monitoring device;
in response to a pre-evacuation condition being satisfied,
determine estimated leakage data based on the region data, the obstacle data, the gas monitoring data, and the environment monitoring data; and
update the safe region and the one or more candidate evacuation paths based on the estimated leakage data.

7. The system of claim 6, wherein the emergency supervision management platform is further configured to:
update an alarm threshold and a monitoring frequency based on the estimated leakage data and one or more updated candidate evacuation paths;
send an alarm control signal to an alarm device deployed in the target region based on an updated alarm threshold, to control the alarm device to perform a setting update based on the updated alarm threshold; and
send a monitoring control signal to the image acquisition device based on an updated monitoring frequency, to control the image acquisition device to perform the setting update based on the updated monitoring frequency.

8. The system of claim 6, wherein the emergency supervision management platform is further configured to:
update the safe region and the one or more candidate evacuation paths based on the estimated leakage data and ventilation device data of the target region.

9. The system of claim 1, wherein the evacuation parameter further includes a modality parameter of the evacuation device, the modality parameter being configured to control a modality setting of the evacuation device; and the emergency supervision management platform is further configured to:
obtain one or more candidate modality parameters;
determine, based on the evacuation path, the region data, the gas monitoring data, and the one or more candidate modality parameters, emergency evacuation data corresponding to each of the one or more candidate modality parameters through a parameter determination model, wherein the parameter determination model is a machine learning model; and
determine the modality parameter based on the emergency evacuation data.

10. The system of claim 9, wherein an input of the parameter determination model includes environment monitoring data of the target region.

11. A method for gas emergency management in an urban public space based on an Internet of Things (IoT) large model, the method being executed by an emergency supervision management platform of a system gas emergency management in the urban public space based on the IoT large model, and the method comprising:
obtaining gas monitoring data of a target region through a monitoring device deployed in a monitoring region based on an emergency supervision object platform;
in response to an evacuation condition being satisfied,
generating an evacuation parameter based on the gas monitoring data and region data of the target region, the evacuation parameter including an evacuation path, an indicator sign control parameter, a lighting control parameter, and a gate control parameter; and
sending an evacuation control signal to an evacuation device deployed in the target region, to control the evacuation device to guide an evacuation of personnel in the target region based on the evacuation parameter, including:
controlling a display device to display the evacuation path;
controlling a display color and an indication direction of an electronic indicator sign based on the indicator sign control parameter;
controlling a lighting brightness and a lighting mode of a lighting device based on the lighting control parameter; and
controlling an on-off state of a gate based on the gate control parameter.

12. The method of claim 11, wherein the monitoring device further includes an image acquisition device, and the generating an evacuation parameter based on the gas monitoring data and region data of the target region includes:
obtaining image sensing data of the target region based on the image acquisition device;
obtaining region personnel data and obstacle data of the target region based on the image sensing data;
determining a safe region and generating one or more candidate evacuation paths based on the gas monitoring data and the region data; and
determining the evacuation path from the one or more candidate evacuation paths based on the region personnel data and the obstacle data.

13. The method according to claim 12, wherein the determining the evacuation path from the candidate evacuation path based on the region personnel data and the obstacle data includes:
obtaining external image data based on the image acquisition device;
determining an evacuation efficiency of each of the one or more candidate evacuation paths based on the region personnel data, the obstacle data, and the region data; and,
determining the evacuation path from the one or more candidate evacuation paths based on the external image data and the evacuation efficiency.

14. The method according to claim 13, wherein the determining an evacuation efficiency of each of the one or more candidate evacuation paths based on the region personnel data, the obstacle data, and the region data includes:
determining the evacuation efficiency of each of the one or more candidate evacuation paths based on the region personnel data, the obstacle data, the region data, and access control data.

15. The method according to claim 12, wherein the monitoring device further includes an environment monitoring device, and the method further comprises:
obtaining environment monitoring data of the target region based on the environment monitoring device;
in response to a pre-evacuation condition being satisfied,
determining estimated leakage data based on the region data, the obstacle data, the gas monitoring data, and the environment monitoring data; and,
updating the safe region and the one or more candidate evacuation paths based on the estimated leakage data.

16. The method according to claim 15, wherein the method further comprises:
updating an alarm threshold and a monitoring frequency based on the estimated leakage data and one or more updated candidate evacuation paths;
sending an alarm control signal to an alarm device deployed in the target region based on an updated alarm threshold, to control the alarm device to perform a setting update based on the updated alarm threshold; and,
sending a monitoring control signal to the image acquisition device based on an updated monitoring frequency, to control the image acquisition device to perform the setting update based on the updated monitoring frequency.

17. The method according to claim 15, wherein the updating the safe region and the one or more candidate evacuation paths based on the estimated leakage data includes:
updating the safe region and the one or more candidate evacuation paths based on the estimated leakage data and ventilation device data of the target region.

18. The method according to claim 11, wherein the evacuation parameter further includes a modality parameter of the evacuation device, the modality parameter being configured to control a modality setting of the evacuation device; and the method further comprises:
obtaining one or more candidate modality parameters;
determining, based on the evacuation path, the region data, the gas monitoring data, and the one or more candidate modality parameters, emergency evacuation data corresponding to each of the one or more candidate modality parameters through a parameter determination model, wherein the parameter determination model is a machine learning model; and
determine the modality parameter based on the emergency evacuation data.

19. The method according to claim 18, wherein an input of the parameter determination model includes environment monitoring data of the target region.

20. A non-transitory computer-readable storage medium, storing one or more computer instructions, wherein when reading the one or more computer instructions in the storage medium, a computer executes a method for gas emergency management in an urban public space based on an Internet of Things (IoT) large model, the method comprising:
obtaining gas monitoring data of a target region through a monitoring device deployed in a monitoring region based on an emergency supervision object platform;
in response to an evacuation condition being satisfied,
generating an evacuation parameter based on the gas monitoring data and region data of the target region, the evacuation parameter including an evacuation path, an indicator sign control parameter, a lighting control parameter, and a gate control parameter; and
sending an evacuation control signal to an evacuation device deployed in the target region, to control the evacuation device to guide an evacuation of personnel in the target region based on the evacuation parameter, including:
controlling a display device to display the evacuation path;
controlling a display color and an indication direction of an electronic indicator sign based on the indicator sign control parameter;
controlling a lighting brightness and a lighting mode of a lighting device based on the lighting control parameter; and
controlling an on-off state of a gate based on the gate control parameter.
